(19) United States
(12) Patent Application Publication
(10) Pub. No.: US 2010/0316184 A1
(43) Pub. Date: Dec. 16, 2010

(54) SILICON PHOTOMULTIPLIER DETECTOR FOR COMPUTED TOMOGRAPHY

(76) Inventors: Jan Iwanczyk, Los Angeles, CA (US); Einar Nygard, Askar (NO); William Barber, Ventura, CA (US); Neal Hartsough, Fullerton, CA (US)

Correspondence Address: CHRISTIE, PARKER & HALE, LLP, PO BOX 7068, PASADENA, CA, 91109-7068, US

(21) Appl. No.: 12/253,901

(22) Filed: Oct. 17, 2008

(51) Int. Cl. G01T 1/208; A61B 6/03

(52) U.S. Cl. 378/19; 250/366

(57) ABSTRACT

An x-ray detector module including a silicon photomultiplier and a computed tomography imaging system including the same is provided. The x-ray detector module includes optically coupled scintillation materials and silicon photomultiplier pixels. The x-ray detector pixels may have a constant axial profile with a polygonal cross section, which may be rectangular. A plurality of the x-ray detector pixels are generally arranged into 2D arrays. Each x-ray detector pixel may be connected to a dedicated electronic readout channel. In operation, the scintillation materials interact with incident x-ray photons to generate visible-light photons. The silicon photomultiplier pixels generate electrical signals in accordance with the number of visible-light photons generated by an incident x-ray photon. The electronic readout channels process the electrical signals to determine characteristics of the incident x-ray photons. Information of a plurality of x-ray photons are compiled to generate computed tomography images.

### BACKGROUND

The present invention relates generally to silicon photomultipliers, and more particularly to the application of silicon photomultipliers in computed tomography, and a computed tomography imaging system including the same.

Silicon Photomultipliers (Si-PMs), also known as Multi-pixel Photon Counters (MPPCs) or Solid State Photomultipliers (SPPMs), are a new type of photon counting device. A Si-PM module is a photon sensitive device which includes one or more Si-PM pixels typically arranged into an array, with each individual Si-PM pixel including a plurality of avalanche photodiodes connected in parallel on a silicon substrate and operated in Geiger mode. Each avalanche photodiode generates a pulse signal when it detects a photon. The pulse signals from the plurality of avalanche photodiodes of a Si-PM pixel are summed to generate an output signal for the Si-PM pixel.

Si-PMs have been considered as potential light signal detectors for applications in the fields of astronomy, astrophysics, high energy physics, and national security, among others. Current application of Si-PMs is still limited, but technological improvements are constantly being realized in Si-PM performance, potentially opening up the application of Si-PMs in more fields.

Computed Tomography (CT) is a primary medical imaging diagnostic modality. Millions of CT procedures are performed in the United States annually. A conventional CT scanner includes x-ray detectors which function in what is referred to as “current-mode.” These x-ray detectors include small blocks of scintillation material, for example, a ceramic scintillation material, coupled to a set of silicon photodiodes. X-ray photons interact with the scintillation material to generate visible-light photons. The visible-light photons interact with the photodiodes to produce an electrical current, the amount of current being proportional to the energy flux of the incoming x-rays. The electrical current is then analyzed by external electronic circuitry, and converted from an analog signal to a digital signal using data acquisition electronics.

Presently, the x-ray detectors in CT scanners operate in an integrating manner, known as current-mode, where the detectors generate signals proportional to the total energy detected as a function of time. Generally, current-mode operation of these x-ray detectors limits the types of information that can be gathered from detected x-rays. For example, specific data or feedback as to the numbers and energy levels of individual x-ray photons cannot be compiled. If obtainable, such information may be utilized in, for example, improvements in imaging quality, reduction in radiation exposure to patients, and more comprehensive tissue color mapping through material decomposition and/or other methods. Furthermore, the ability to identify low level contrast agents introduced into a patient can expand CT imaging from anatomical to combined anatomical and functional imaging modalities.

In an effort to overcome limits imposed by current-mode detectors, a number of research groups in hospitals, universities and commercial companies are actively engaged in developing energy-dispersive “photon counting” detector systems for CT imaging applications. Until recently, only direct conversion cadmium zinc telluride (CdZnTe) and cadmium telluride (CdTe) compound semiconductor detectors were seriously considered as possible candidates for photon counting detectors in CT scanners.

### SUMMARY OF THE INVENTION

An exemplary embodiment of the present invention provides for photon detectors utilizing silicon photomultipliers, and a computed tomography imaging system including the same. In one aspect, an exemplary embodiment of the present invention provides an x-ray detector module including: an array of scintillation crystals; an array of silicon photomultiplier pixels optically coupled to the scintillation crystals, each of the scintillation crystals corresponding to at least one of the silicon photomultiplier pixels; and an array of electronic readout channels coupled to the silicon photomultiplier pixels, each of the silicon photomultiplier pixels being coupled to a corresponding one of the electronic readout channels, wherein an x-ray detector pixel includes a one of the scintillation crystals and a corresponding one of the silicon photomultiplier pixels, and wherein each of the x-ray detector pixels and corresponding electronic readout channel is configured to support a count rate up to at least 10⁶ detected x-ray photons per second.

In another aspect, an exemplary embodiment of the present invention provides an x-ray detector system including: a plurality of x-ray detector pixels, each of the x-ray detector pixels including a scintillation material for detecting x-ray photons and generating light photons in accordance with the detected x-ray photons, and a silicon photomultiplier pixel optically coupled to the scintillation material for generating electrical signals corresponding to a number of the generated light photons; a corresponding electronic readout channel coupled to each of the silicon photomultiplier pixels for sorting and counting the electrical signals; and a processor configured by program instructions to generate images in accordance with the detected x-ray photons, the program instructions including instructions for compiling information of the detected x-ray photons and the corresponding x-ray detector pixels to generate the images.

In yet another aspect, an exemplary embodiment of the present invention provides a method of detecting an x-ray photon in computed tomography, including: generating a plurality of light photons from interaction of the x-ray photon with a scintillation material; generating an electrical signal corresponding to a number of the plurality of light photons by utilizing a silicon photomultiplier pixel; and determining an energy of the x-ray photon based on a magnitude of the electrical signal.

These and other aspects of the present invention are more fully comprehended upon review of this disclosure.

### BRIEF DESCRIPTION OF THE DRAWINGS

FIG. 1 is an example of a block diagram of an x-ray photon detector system for computed tomography applications in accordance with aspects of an embodiment of the present invention.

FIG. 2 is an illustration of an x-ray detector pixel in accordance with aspects of an embodiment of the present invention.

FIG. 3 illustrates a schematic top view of a Si-PM pixel in accordance with aspects of an embodiment of the present invention.

FIGS. 4A and 4B are block diagrams illustrating embodiments of an electronic readout channel in accordance with aspects of an embodiment of the present invention.

FIG. 5 is an illustration of an assembly for a computed tomography detector system in accordance with aspects of an embodiment of the present invention.

FIGS. 6A, 6B, and 6C illustrate various views of a printed circuit board including a double-sided ball grid array in accordance with aspects of an embodiment of the present invention.

### DETAILED DESCRIPTION

In the following detailed description, certain exemplary embodiments of the present invention are shown and described by way of illustration. As those skilled in the art would realize, the described embodiments may be modified in various different ways, without departing from the spirit or scope of the present invention. Accordingly, the drawings and description are to be regarded as illustrative in nature and not restrictive.

In exemplary embodiments of the present invention, an x-ray detector module includes a plurality of x-ray detector pixels, each x-ray detector pixel including a scintillation material and a silicon photomultiplier (Si-PM) pixel having a plurality of avalanche photodiodes (APDs). X-ray detector modules further include at least one electronic readout channel. The detector module is utilized to maintain a spatial relationship of the radiation entering an imaging detector in a computed tomography (CT) application and/or other applications involving x-ray imaging or detection. Information may be provided about a number of detected x-ray photons and the individual energy levels of each of the detected x-ray photons. A scintillation material is physically and optically aligned with a corresponding Si-PM pixel to form an x-ray detector pixel for detecting x-ray photons. Visible light photons are generated from the interaction of x-ray photons with the scintillation material, and the plurality of APDs of an individual Si-PM pixel detect and sum the number of visible-light photons generated. The output of the Si-PM pixel is connected to a single electronic readout channel. Determination of the energy of the original x-ray photon is based on the summation.

In an exemplary embodiment of the present invention, the imaging of very high x-ray photon flux is accomplished through utilization of the high speed and high light sensitivity of Si-PM operations combined with fast scintillators and high-speed parallel-channel readout electronics. The detector system has a high dynamic range for x-ray photon counting, having the capability of detecting at least 10⁶ x-ray photons per mm² per second in one embodiment. In other embodiments, the upper limit of the dynamic range for x-ray photon counting may be on the order of 10⁷ or 10⁸ x-ray photons per mm² per second. Therefore, the scintillation materials utilized have very fast decay times and high light yield emissions, and the Si-PM pixels have high light sensitivity and extremely fast response times. As such, electrical signals may be generated in response to incident x-ray photons fast enough so as to differentiate between two consecutive x-ray photon interactions in a single detector pixel.

In another exemplary embodiment of the present invention, the detection and imaging system, according to an embodiment of the present invention, includes an application specific integrated circuit (ASIC), which is utilized for signal amplification and signal processing electronics. In exemplary embodiments, the ASIC may contain electronic readout channels organized in a parallel configuration, with each of the electronic readout channels dedicated to a corresponding one of the x-ray detector pixels, and a single common I/O controller. Such a configuration provides for faster processing times, as one electronic readout channel does not receive multiplexed signals, and does not need to separate and/or distinguish signals from multiple x-ray detector pixels. Successful photon counting at the high levels of x-ray flux involved in CT applications reduces the applicability of multiplexing, due to, for example, processing speed constraints. Although multiplexing is commonly used in imaging detectors for positron emission tomography (PET) and single photon emission computed tomography (SPECT), multiplexers hinder signal processing speeds, and would therefore reduce the effectiveness of the CT imaging detectors. Therefore, multiplexers are not used in some embodiments of the present invention. In other embodiments of the present invention, multiplexing may be utilized if it is determined, for example, that multiplexing would not reduce the efficiency and processing speed of the CT detector system.

Each component of the x-ray detector pixel is configured to have a full vertical integration. In exemplary embodiments of the present invention, the components of the x-ray detector pixel have four-side buttability, such that each x-ray detector pixel is capable of being aligned with adjacent x-ray detector pixels on all four sides, with very small amounts of dead space between adjacent x-ray detector pixels. Arrangement of x-ray detector pixels into 2D matrices or arrays allows for multislice CT configurations without imposing limitations on the number of slices that can be utilized.

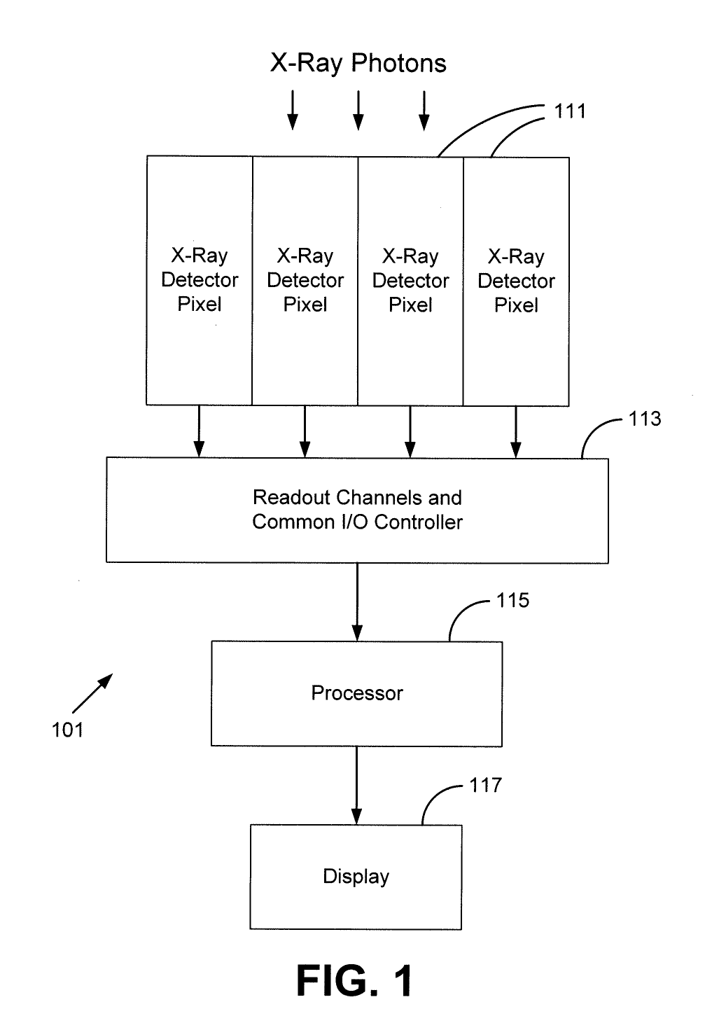

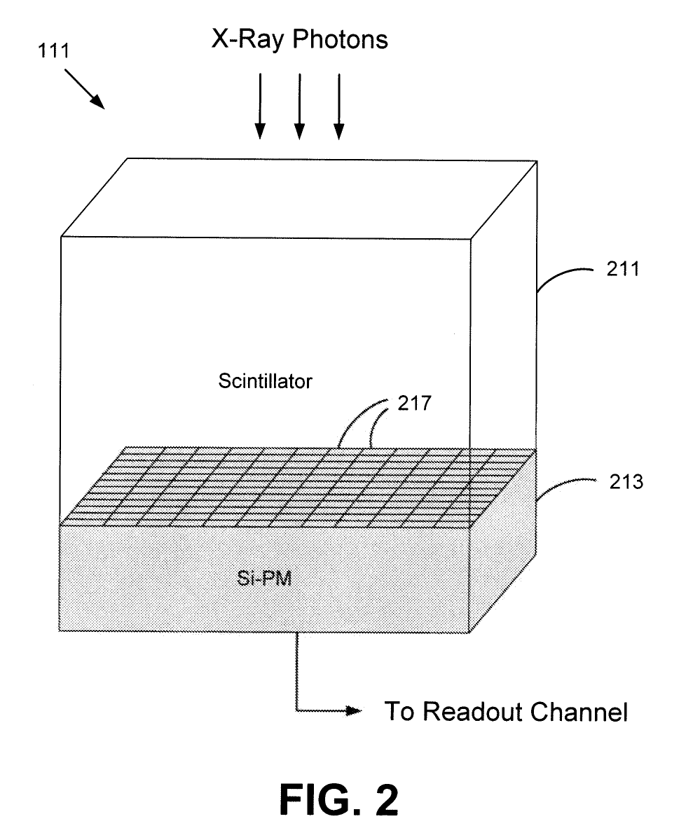

FIG. 1 is an example of a photon counting CT detector system 101 in accordance with aspects of the present invention, and FIG. 2 is an example of an x-ray detector pixel 111 in FIG. 1.

In exemplary embodiments, the CT detector system 101 includes a plurality of x-ray detector pixels, for example, x-ray detector pixels 111, a plurality of corresponding readout channels with a common input/output (I/O) control 113, a processor 115, and a display 117. In alternate embodiments, more or fewer components may be included in the CT detector system. For example, the processor 115 may be included in a larger computing system or a more complex imaging or output system may be included in addition to, or in lieu of, the display 117.

In FIG. 1, the x-ray detector pixels 111 are arranged in a module or array format. While only four x-ray detector pixels are illustrated in the detector module of FIG. 1 for convenience of description, in practice, a detector module may include more or fewer x-ray detector pixels. In practice, similar x-ray detector pixels may be arranged and provided in 2D arrays in similar configurations, with an exemplary detector module including, for example, a package having a rectangular grid including a plurality of aligned x-ray detector pixels. In some embodiments, a plurality of x-ray detector pixels are provided on a common substrate in an array configuration. Upon receipt of an incident x-ray photon, one of the x-ray detector pixels 111 detects the x-ray photon, and generates an electrical signal based on the energy of the detected x-ray photon.

Conventional current-mode CT detectors are limited in determining energies of detected x-ray photons, due to property limitations and/or physical limitations of the photodiodes and scintillation materials used. To overcome the limitations of current-mode CT detectors, the x-ray detector pixels 111 facilitate more comprehensive x-ray photon detection by utilizing the Si-PMs and new faster scintillation materials to realize energy-dispersive photon detection and counting. A CT detector system should be capable of very high x-ray photon count rates to handle the high count rate dynamic range of CT applications, with processing times often on the order of nanoseconds. High x-ray photon count rates provide more accurate statistical information during a scan of moving organs, for example, the heart, in a very short time interval, such that the movement of the organs during the scan is negligible, thus reducing image blurring due to organ movement. Improved energy resolution provided by the present invention, and which is not present in current-mode detectors, allows for the detection and differentiation of the different energies of the incident x-ray photons.

The x-ray detector pixels 111 send signals to corresponding readout channels of the readout channels and common I/O controller 113. After the signals are analyzed by the readout channels, information about the signals is sent through a common I/O controller to the processor 115 for processing. In some embodiments, the information may be in the form of electrical signals representing the response of the x-ray detector pixels to different x-ray photons. In some embodiments, the information may be in the form of counts from one or more energy-selective counters of the electronic readout channels. The electronic readout channels may be arranged on an ASIC interface customized for the CT detector system. An ASIC may include a plurality of parallel electronic readout channels, with each x-ray detector pixel coupled to a corresponding one of the plurality of parallel electronic readout channels on the ASIC. The processor may include, for example, image processors and/or a computer system, to process images.

The processor 115 provides processed signals to a display or similar output device 117. In some embodiments, the display device may include a conventional computer display, a television display, or a display integrated with the processor. In other embodiments, the display device may include a data readout device which provides, for example, compilation of different statistics, or for example, a printer for printing the statistics. The images or information may include, for example, images representative of a distribution of x-ray intensities detected by the x-ray detector pixels, or for example, distributions of the energies of detected x-ray photons impinging upon the imaging detector system 101, or both. These and other similar forms of more detailed x-ray information may be utilized to enhance resulting images.

For a CT detector system to be considered effective, the x-ray detector pixels should be able to detect low-energy x-ray photons, of approximately 30 keV, with sufficient signal-to-noise ratio, while also having good detection efficiency at high-energy x-ray photon levels, up to about 140 keV. The x-ray detector pixels should further be able to accommodate very high count rates, have good energy resolution, and have good response. Furthermore, the form factor of the x-ray detector pixels should be sufficiently small to be capable of supporting image pixels having sub-millimeter dimensions, and should also be able to operate at room temperature. In exemplary embodiments, x-ray detector pixels should also be easily bonded to substrates and processing electronics.

FIG. 2 is an example of a structure of an x-ray detector pixel, such as the x-ray detector pixel 111 described with respect to FIG. 1. The x-ray detector pixel 111 of FIG. 2 includes a scintillation material 211 and a Si-PM pixel 213.

The x-ray detector pixel 111 utilizes the scintillation material 211 and the Si-PM pixel 213 to maintain a spatial relationship of the radiation entering the imaging detector and to provide information about the actual number of x-ray photons and the energies of each of the x-ray photons. In some embodiments, the scintillation material may be a scintillation crystal segment. In some embodiments, the scintillation material may include numerous segments. The Si-PM pixel includes a plurality of APDs, for example, APDs 217, arranged in an array, where each of the APDs is configured to detect visible-light photons produced by an interaction of x-ray photons with the scintillation material. In some embodiments, optical reflectors between the scintillation segments reflect light back to the segments, reducing escape of the visible-light photons from the scintillation segments. The scintillation crystal segments are optically coupled to the Si-PM pixels by an optically transparent layer. The refractive index of the coupling layer is generally chosen to minimize or reduce light loss by internal reflections at the interface between the scintillation material and the Si-PM pixel. In some embodiments, the coupling layer is a transparent epoxy layer.

In operation, interaction between an incident x-ray photon and the scintillation material of an x-ray detector pixel generates a plurality of visible-light photons, the number of the visible light photons based on the energy of the incident x-ray photon. For example, a higher energy x-ray photon may interact with the scintillation material to generate a greater number of visible-light photons than a lower energy x-ray photon. In some embodiments, an anti-scatter grid may be co-registered or included with the scintillation crystal segments to reduce or minimize the effect of x-ray scatter from an imaged subject. The visible-light photons associated with an incident x-ray photon are detected concurrently by the plurality of APDs of the Si-PM pixel, and an electrical signal having a magnitude corresponding to an aggregate number of detected visible-light photons by all the APDs of a Si-PM pixel is transmitted to a corresponding electronic readout channel. The electronic readout channel may process the signal, for example, by incrementing an appropriate counter corresponding to a magnitude of the electrical signal.

In CT scanning, since incident x-ray photons reach the CT detector system at very high rates, up to more than 10⁶ x-ray photons per mm² per second, and in some embodiments, more than 10⁷ or 10⁸ x-ray photons per mm² per second, and since x-ray detector pixels in this invention may be configured to detect these high numbers of x-ray photons in a very short period of time, the CT detector system should be configured to process the detection of x-ray photons at very fast speeds. The imaging of high x-ray photon flux utilizing photon counting and energy discrimination with scintillation materials optically coupled to Si-PM pixels may be realized through utilizing new scintillation materials with very fast decay times and high light yield emissions and new Si-PM devices with high light sensitivity and very fast response times. Scintillation materials with high light yield emissions generate a high number of light photons per unit energy of an incident x-ray photon, have fast decay times generally less than 100 nanoseconds, and provide quick processing and decay of the light photon flux prior to subsequent incident x-ray photons. Si-PM pixels with high light sensitivity detect light photons efficiently and accurately, and fast response times allow corresponding readout channels to distinguish between visible-light photons generated by a previous x-ray photon and visible-light photons generated by a subsequent x-ray photon.

In recent years several potential scintillation materials have been developed, such as, for example, cerium doped lutetium orthosilicate (LSO), cerium doped lutetium yttrium orthosilicate (LYSO), ytrium aluminum perouskite (YAP), lutetium aluminum perovskite (LuAP), and cerium doped lanthanum bromide (LaBr), among others, for many applications. Compared with previously developed scintillation materials, these new scintillation materials have relatively high light output, very fast scintillation decay, and high atomic number or high density. For example, in the above scintillation materials, light yielded from interaction with x-ray photons range from 10 photons/keV of an x-ray photon to 61 photons/keV of an x-ray photon. Decay times range from 18 nanoseconds to 48 nanoseconds, much faster than previously developed and more widely used scintillation materials, for example, thalium doped sodium iodide (NaI:Tl) scintillation materials, which have decay times on the order of approximately 230 nanoseconds. The decay time improvements of the new scintillation materials are significant in that the new scintillation materials make detection and processing by readout electronics at count rates of 10⁷ to 10⁸ x-ray photons per second feasible. There is ongoing research into the development of newer scintillation materials with even higher light yields and faster decay times, which may be better suited for application in a CT detector system.

Furthermore, Si-PM pixels offer improved performance characteristics over, for example, conventional photomultiplier tubes, conventional semiconductor photodiodes, and conventional avalanche photodiodes. For example, Si-PM pixels provide very fast signal response times and are responsive to single visible-light photons. Si-PMs have high signal gain with low noise, are operable with a low bias voltage, have low power consumption, and are compact and robust. In addition, Si-PM pixels operate efficiently at room temperature and are insensitive to magnetic fields.

The x-ray detector pixel 111 of FIG. 2 is connected to one of a number of electronic readout channels. The electronic readout channel converts signals generated by the Si-PM pixel 213 to information useable by the processor 115 of FIG. 1. As described above with respect to FIG. 1, the electronic readout channel may be included in an ASIC interface. An ASIC may include parallel electronic readout channels which each include, for example, amplifiers, shapers, discriminators, counters, analog-to-digital converters, and other components, some of which may be common to all readout channels for input/output (I/O) control.

In some embodiments, the x-ray detector pixel 111 is configured to have full vertical integration. That is, the various components of an x-ray detector pixel are shaped such that the vertical profile of the x-ray detector pixel is substantially consistent, and cross-sections of the x-ray detector pixel perpendicular to the incident direction of x-rays are substantially constant along the length of the x-ray detector pixel. Generally, the cross-sections of the x-ray detector pixel are polygonal in shape. Further, in many instances, the x-ray detector pixel is configured to have four-side buttability. That is, the cross-section of the x-ray detector pixel is generally configured to be rectangular in shape, to provide for ease of alignment of multiple adjacent x-ray detector pixels in a detector system. Such a configuration allows for tiling of the x-ray detector pixels into a planar array, where each x-ray detector pixel may be surrounded on up to four sides by other identical x-ray detector pixels with minimal or a very small amount of space between adjacent x-ray detector pixels. Such an arrangement provides for a CT detector system where the x-ray detector pixels are arranged in a multi-module 2D matrix. In some embodiments, full vertical integration may be achieved using alternative detector configurations, for example, x-ray detector pixels having three-side buttability, or for example, x-ray detector pixels having six-side buttability, depending on the particular embodiment. Full vertical integration of the x-ray detector pixels provides for a multislice CT configuration without imposing limitations on the number of slices that may be used.

In some embodiments, individual detectors or the detector system includes a temperature monitor or controller for maintaining the detector system at an optimal temperature, for example, above or below room temperature. Such temperature stabilization may reduce electronic noise and stabilize detector performance and response.

In some embodiments a similar temperature control and/or feedback system is utilized to control a voltage supplied to the x-ray detector pixels, to maintain, for example, stable operating conditions for the CT detector system. For example, the voltage could be controlled to maintain a constant internal gain of the APDs, to compensate or adjust for gain changes in APDs due to temperature variations.

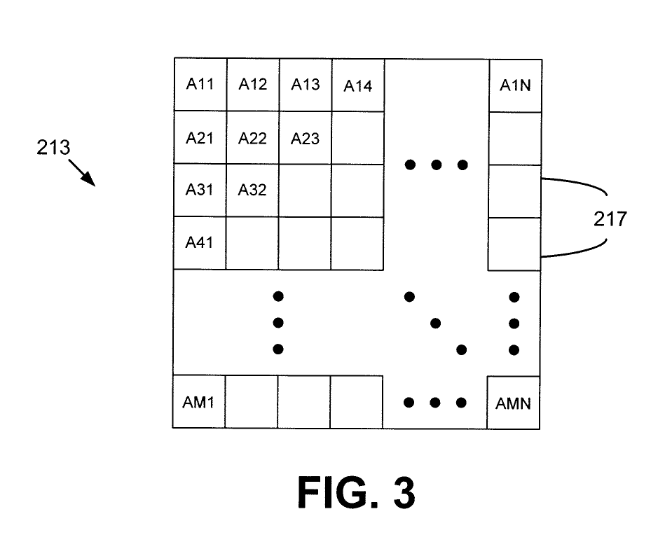

FIG. 3 is a schematic top view of an example of a Si-PM pixel in accordance with aspects of the present invention. In FIG. 3, a Si-PM pixel 213 of an x-ray detector pixel, such as the x-ray detector pixel 111 in FIGS. 1 and 2, includes a plurality of APDs, for example, APDs 217, arranged in a 2D array. The Si-PM pixel 213 is arranged such that there are N number of APDs per row and M number of APDs per column, forming an M×N array of APDs in each Si-PM pixel.

In some embodiments, an x-ray detector pixel 111 may have an approximate size of, for example, 1 mm×1 mm, with for example, a Si-PM pixel having 400 APDs, each having a size of 50 μm×50 μm. Another embodiment may utilize APDs having a size of 100 μm×100 μm, such that each detector pixel has 100 APDs. In each of these embodiments, the APDs, with corresponding quenching resistors, are generally connected in parallel, such that an electrical signal generated by a Si-PM pixel is a summation of the individual signals generated by each APD. Various different embodiments of Si-PM pixels have between 100 and 5000 APDs, each APD sized equal to or smaller than 100 μm×100 μm. The Si-PM pixels may be formed on a common substrate. In exemplary embodiments, a Si-PM pixel is generally limited to sizes equal to or smaller than 1 mm×1 mm, depending on the desired spatial resolution of the CT detector system.

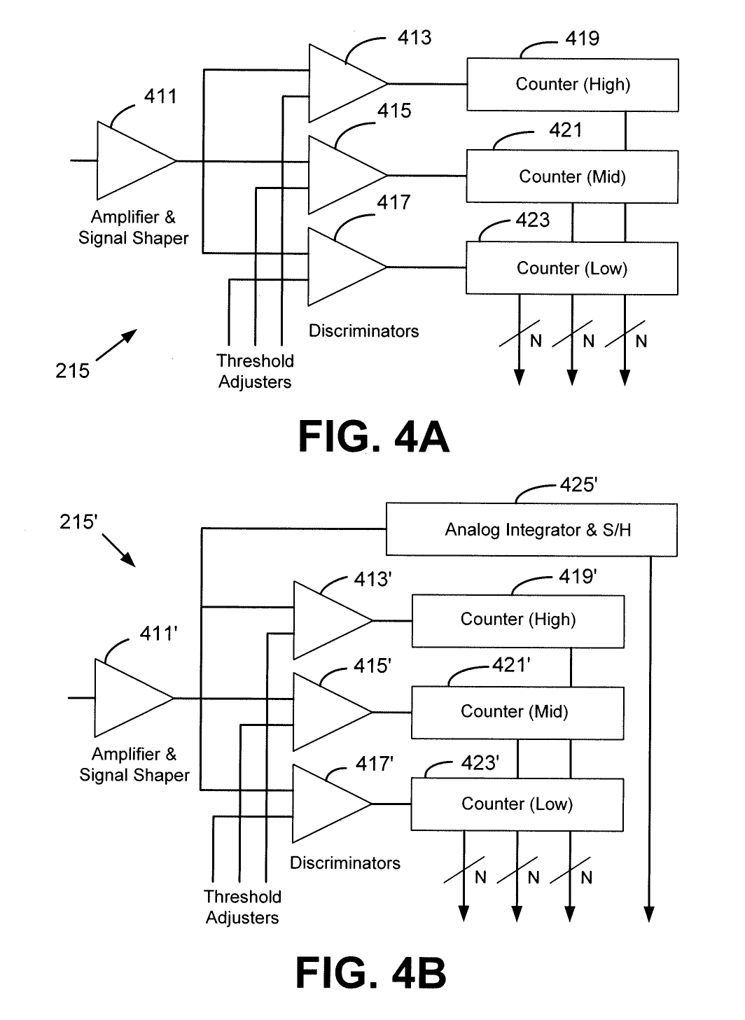

FIGS. 4A and 4B illustrate exemplary schematic block diagrams of individual electronic readout channels, for example, one of a plurality of individual electronic readout channels included in the readout channels and common I/O controller 113 of FIG. 1. In some embodiments, a separate individual electronic readout channel is coupled to each x-ray detector pixel in a CT detector, and the individual electronic readout channels are arranged in parallel on an ASIC interface for delivering information to a processor. Referring to FIG. 4A, in one embodiment, an individual electronic readout channel 215 includes an amplifier and signal shaper 411, a plurality of discriminators, for example, discriminators 413, 415, and 417, and a plurality of counters, for example, counters 419, 421, and 423. In exemplary embodiments, each individual electronic readout channel includes at least two discriminators and at least two counters, to distinguish between at least an electrical signal corresponding to a higher energy x-ray photon and a lower energy x-ray photon.

In operation, the individual electronic readout channel receives an electrical signal generated by a corresponding Si-PM pixel in response to an incident x-ray photon. In many embodiments, the electrical signal is received by an amplifier and signal shaper 411 to amplify and shape the electrical signal to assist in detection and discrimination.

A plurality of discriminators are connected to the amplifier and signal shaper 411. In the embodiment of FIG. 4A, three discriminators 413, 415, and 417 are connected to the amplifier and signal shaper. In other embodiments, more or less than three discriminators may be implemented into an individual electronic readout channel, depending on the particular embodiment. The discriminators sort the electrical signal into energy bins or categories based on the energy level or the magnitude of the electrical signal. For example, in the embodiment illustrated in FIG. 4A, a first discriminator 413 may accept electrical signals representing a relatively higher energy level, a second discriminator 415 may accept electrical signals representing a mid level energy level, and a third discriminator 417 may accept electrical signals representing a relatively lower energy level. This process may help determine an approximation of the energy level of the originating x-ray photon, which may be between, for example, 30 keV and 140 keV.

In many embodiments, the selection of the energy levels of the discriminators may be adjusted by adjusting threshold adjusters of each individual discriminator. Threshold level selection and adjustment may be done externally through, for example, software by utilizing a computer system. Therefore, the individual electronic readout channels in the CT detector system may be adjusted, making the CT detector system more versatile. For example, threshold adjustments may be made to accommodate different imaging tasks or to account for manufacturing variations of scintillation material light yields from one pixel to another.

Each of the discriminators is connected to a counter, which counts a number of electrical signals sorted and transmitted through the corresponding discriminator. For example, in the embodiment of FIG. 4A, the individual electronic readout channel 215 includes a discriminator 413 connected to a counter 419 for high energy level electrical signals, the discriminator 415 is connected to a counter 421 for mid energy level electrical signals, and the discriminator 417 is connected to a counter 423 for low energy level electrical signals. In other embodiments with more or less number of discriminators, a separate corresponding counter is generally connected to each discriminator. The counters count the number of electrical signals sorted by the respective discriminator, and transmits signals based on the number of detected electrical signals to a processor. The processor receives the signals from each of the counters, and determines numbers and energy levels of detected x-ray photons by each corresponding x-ray detector pixel in the CT detector system.

Referring now to FIG. 4B, in an alternate embodiment, an individual electronic readout channel 215′ is configured similarly to the individual electronic readout channel 215 described in reference to FIG. 4A above. The individual electronic readout channel 215′ in FIG. 4B similarly includes an amplifier and signal shaper 411′, a plurality of discriminators, for example, discriminators 413′, 415′, and 417′, and a plurality of counters, for example, counters 419′, 421′, and 423′. The individual electronic readout channel of FIG. 4B further includes an additional analog integrator 425′ connected to the amplifier and signal shaper 411′, and in parallel with the discriminators 413′, 415′, and 417′. In exemplary embodiments, the analog integrator 425′ may include its own sample/hold circuitry. The analog integrator may allow the CT detector to operate in ranges beyond saturation or similar operating limits of the other electronics or components of the CT detector. The analog integrator may be used to extend a dynamic range of the CT detector to detect, for example, higher count rates, or for example, a higher range of x-ray flux.

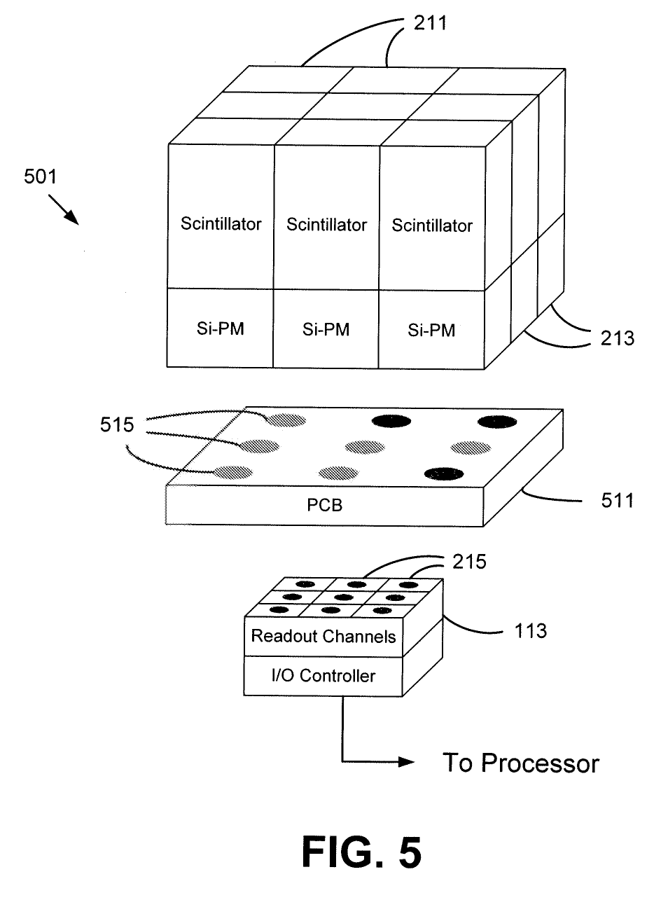

FIG. 5 is an illustration of a CT detector module in accordance with aspects of the present invention. The CT detector module 501 includes a plurality of optically coupled scintillation crystal 211 and Si-PM pixel 213 assemblies arranged in a 2D array, a printed circuit board insert 511, and an ASIC 113 including a plurality of individual electronic readout channels, for example, individual electronic readout channels 215, which may be similar to the individual electronic readout channels described above with respect to FIGS. 4A and 4B, and a common I/O controller.

Generally, scintillation crystal/Si-PM pixel assemblies in accordance with aspects of the present invention interface with an ASIC, which includes the individual electronic readout channels described above, prior to communication with a processor. A scintillation crystal/Si-PM pixel assembly may be sized much larger than a typical individual electronic readout channel on an ASIC. Generally, absent major modifications to ASIC manufacturing, potential CT detector module arrangements may be limited by manufacturing limitations of the ASIC, which may consequently limit the dimensions of a 2D array, or potentially limit or alter vertical integration of the 2D array.

In the embodiment of FIG. 5, the printed circuit board insert may serve as a pitch adapter, allowing for interconnections of larger scintillation crystal/Si-PM pixel assemblies to smaller sized individual readout channels on an ASIC, while preserving the true vertical integration of the x-ray detector pixels. In some embodiments, the printed circuit board insert 511 may serve a dual role and constitute a double-sided ball grid array package for the ASIC, including contacts, for example, contacts 515, to ease connectivity with both the scintillation crystal/Si-PM pixel assemblies and the ASIC. An example of a double-sided ball grid array is disclosed in U.S. Pat. No. 7,170,049 issued Jan. 30, 2007, the entire content of which is incorporated by reference herein.

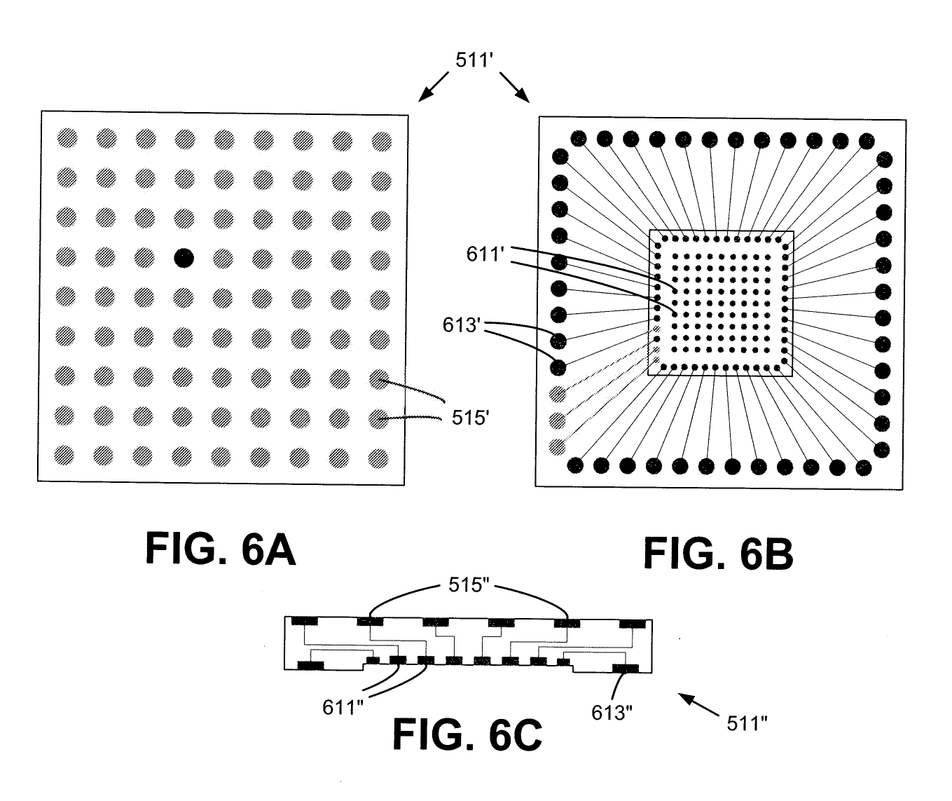

An example of a printed circuit board including a double-sided ball grid array in accordance with aspects of embodiments of the present invention will now be discussed in further detail, with reference to FIGS. 6A, 6B, and 6C.

FIG. 6A is a top view of a first side of a printed circuit board including a double-sided ball grid array, and FIG. 6B is a top view of a second side of the printed circuit board of FIG. 6A. FIG. 6C is an example of a cross-section of a printed circuit board in exemplary embodiments.

The printed circuit board 511′ may be similar to the printed circuit board 511 discussed with respect to FIG. 5. In exemplary embodiments of the invention, the first side of the printed circuit board 511′ may correspond to a side which connects to an array of Si-PM pixels, and the second side of the printed circuit board 511′ may correspond to a side which connects to an ASIC.

Referring to FIG. 6A, the printed circuit board 511′ includes an array of solder balls or conductive epoxy 515′, which serve as contact electrodes, generally for connecting to Si-PM pixels. In exemplary embodiments of the invention, a separate contact electrode is available for each Si-PM pixel connected to the printed circuit board, whereby an entire CT detector module may include one or more printed circuit boards connected to one or more processors.

Referring now to FIG. 6B, a reverse side of the printed circuit board 511′ may include a smaller array of contacts, for example, contact electrodes 611′ for connecting to an ASIC or similarly configured circuitry. The printed circuit board 511′ may serve as a pitch adapter for interconnecting the Si-PM pixels to a circuit having a smaller form factor, such as an ASIC, while allowing the detector pixels to maintain array configurations as have been described above. In some embodiments, the contacts 611′ are smaller than the contacts 515′ of FIG. 6A. In some embodiments, the contacts 611′ may be arranged similarly to the contacts 515′, while taking up less space on the printed circuit board. In some embodiments, an area in which contacts 611′ are located may be recessed from a surface of the face of the printed circuit board 511′, to accommodate an appropriately sized ASIC or similar circuitry, as is discussed in more detail with respect to FIG. 6C below. The second face of the printed circuit board 511′ also includes a plurality of service pad contacts, for example, service pads 613′, which may provide power and/or facilitate communication with a larger processor, for example, to a motherboard housing further components and/or circuitry. In some embodiments, the service pads 613′ may be located in edge regions of the printed circuit board 511′, and may interconnect to a central portion of the printed circuit board where an ASIC or similar circuitry is located, to provide the ASIC with a means of communication to an external processor or motherboard.

FIG. 6C is a schematic example of a cross-sectional view of a printed circuit board in accordance with aspects of the invention. The configuration of the printed circuit board 511″ of FIG. 6C may be similar to the configuration of the printed circuit board 511′ described with respect to FIGS. 6A and 6B. The printed circuit board 511″ of FIG. 6C includes a plurality of contact electrodes 515″ on a first side of the printed circuit board, and a plurality of contact electrodes 611″ and service pads 613″ on a second side of the printed circuit board. Generally, each of the plurality of contact electrodes 515″ is connected through the printed circuit board to a corresponding one of the plurality of contact electrodes 611″. In some embodiments, the contact electrodes 611″ may be located on a surface recessed from a main surface of the second face to more easily accommodate an ASIC or similar chip or circuitry, such that a surface formed by the second side and a face of an attached ASIC may be substantially flat. In this manner, such a combination may be more easily integrated onto different motherboards in different CT detector system embodiments, where service pads 613″ may more easily connect to a surface or pins of an adjoining board or circuit. A recessed surface may also prevent or reduce the possibility of connecting an incompatible or incorrect ASIC or similar chip or circuitry not intended for use with the printed circuit board or the detector system, or of installing a correct chip or circuitry in an incorrect manner.

Thus, a printed circuit board insert, for example, the printed circuit boards including double-sided ball grid arrays described with respect to FIGS. 6A, 6B, and 6C, may be integrated into an assembly, for example, the CT detector system assembly of FIG. 5, to more readily interconnect individual Si-PM pixels to an ASIC or similar circuitry.

In embodiments described above with respect to the present invention, the output signal from each Si-PM pixel of an x-ray detector pixel 111 is transmitted to a corresponding electronic readout channel. In some embodiments, the electronic readout channels may be formed on an ASIC. In some embodiments, an ASIC electronics configuration may contain up to between 128 and 1024 electronic readout channels in a single monolithically integrated chip. Therefore, a large number of Si-PM pixels may be connected and/or controlled through a single ASIC, thus reducing the number of ASICs manufactured for each complete CT detector system.

The present invention therefore provides for x-ray detector pixels utilizing silicon photomultipliers, and a computed tomography or other x-ray imaging application including the same. Although the present invention has been described in connection with certain embodiments, it should be recognized that the present invention is not limited to the embodiments specifically described, but is instead intended to comprise the various modifications and equivalent arrangements which are included within the spirit and the scope of the appended claims and their equivalents.

What is claimed is:

1. An x-ray detector module comprising:
an array of scintillation crystals;
an array of silicon photomultiplier pixels optically coupled to the scintillation crystals, each of the scintillation crystals corresponding to at least one of the silicon photomultiplier pixels; and
an array of electronic readout channels coupled to the silicon photomultiplier pixels, each of the silicon photomultiplier pixels being coupled to a corresponding one of the electronic readout channels,
wherein an x-ray detector pixel comprises a one of the scintillation crystals and a corresponding one of the silicon photomultiplier pixels, and
wherein each of the x-ray detector pixels and corresponding electronic readout channel is configured to support a count rate up to at least 10⁶ detected x-ray photons per second.

2. The x-ray detector module of claim 1, wherein the array of electronic readout channels comprise at least one application specific integrated circuit.

3. The x-ray detector module of claim 1, wherein an axial cross-section of each x-ray detector pixel is a polygon comprising at least three sides, and wherein each of the at least three sides of the x-ray detector pixel is a flat surface and is aligned opposite a corresponding side of an adjacent x-ray detector pixel.

4. The x-ray detector module of claim 3, wherein the axial cross-section of each x-ray detector pixel is rectangular and is equal to or smaller than 1 mm×1 mm.

5. The x-ray detector module of claim 1, wherein each of the silicon photomultiplier pixels is configured to generate an electric signal in response to each detected x-ray photon.

6. The x-ray detector module of claim 1, wherein an energy of each detected x-ray photon is determined, and wherein the electronic readout channels are configured to catalog the detected x-rays photons based on the determined energies.

7. The x-ray detector module of claim 1, wherein each of the silicon photomultiplier pixels is optically coupled to the corresponding one of the scintillation crystals via an epoxy layer.

8. The x-ray detector module of claim 1, wherein each of the x-ray detector pixels and corresponding electronic readout channel is configured to support a count rate up to at least 10⁷ detected x-ray photons per second.

9. The x-ray detector module of claim 1, wherein each of the x-ray detector pixels and corresponding electronic readout channel is configured to support a count rate up to at least 10⁸ detected x-ray photons per second.

10. The x-ray detector module of claim 1, wherein the scintillation crystals have a principal decay time of less than 100 nanoseconds.

11. The x-ray detector module of claim 1, wherein the scintillation crystals comprise at least one of cerium doped lutetium orthosilicate (LSO), cerium doped lutetium yttrium orthosilicate (LYSO), ytrium aluminum perouskite (YAP), lutetium aluminum perovskite (LuAP), or cerium doped lanthanum bromide (LaBr).

12. An x-ray detector system comprising:
a plurality of x-ray detector pixels, each of the x-ray detector pixels comprising a scintillation material for detecting x-ray photons and generating light photons in accordance with the detected x-ray photons, and a silicon photomultiplier pixel optically coupled to the scintillation material for generating electrical signals corresponding to a number of the generated light photons;
a corresponding electronic readout channel coupled to each of the silicon photomultiplier pixels for sorting and counting the electrical signals; and
a processor configured by program instructions to generate images in accordance with the detected x-ray photons, the program instructions including instructions for compiling information of the detected x-ray photons and the corresponding x-ray detector pixels to generate the images.

13. The x-ray detector system of claim 12, wherein an axial cross-section of each of the plurality of x-ray detector pixels is rectangular and is substantially constant for an axial length of the x-ray detector pixel.

14. The x-ray detector system of claim 13, wherein the axial cross-section of each of the plurality of x-ray detector pixels has a size equal to or smaller than 1 mm×1 mm.

15. The x-ray detector system of claim 12, wherein each of the electronic readout channels comprises:
an amplifier for amplifying the electrical signals;
at least two discriminators for sorting the electrical signals in accordance with threshold settings of the at least two discriminators; and
at least two counters, each of the at least two counters coupled to a corresponding one of the at least two discriminators for counting signals sorted by the corresponding one of the at least two discriminators.

16. The x-ray detector system of claim 15, wherein each of the electronic readout channels further comprises an analog integrator for extending a dynamic range of the electronic readout channel.

17. The x-ray detector system of claim 12, wherein each of the x-ray detector pixels further comprises an anti-scatter grid.

18. The x-ray detector system of claim 12, wherein the electronic readout channels for at least two of the plurality of x-ray detector pixels are located on an application specific integrated circuit (ASIC) chip.

19. The x-ray detector system of claim 18, further comprising a printed circuit board, wherein an area of the ASIC chip is smaller than a combined cross-sectional area of the silicon photomultiplier pixels of the at least two of the plurality of x-ray detector pixels, and wherein the printed circuit board comprises:
a double-sided ball grid array for electrically coupling the silicon photomultiplier pixels of the at least two of the plurality of x-ray detector pixels with the ASIC chip; and
a plurality of service pads for electrically coupling the ASIC chip to the processor.

20. The x-ray detector system of claim 18, wherein the ASIC chip comprises between 128 and 1024 electronic readout channels.

21. The x-ray detector system of claim 12, further comprising a temperature controller for controlling a temperature of at least one of the plurality of x-ray detector pixels or the electronic readout channels.

22. The x-ray detector system of claim 12, further comprising a voltage controller for controlling a voltage supplied to at least one of the plurality of x-ray detector pixels or the electronic readout channels according to temperature changes of the at least one of the plurality of x-ray detector pixels or the electronic readout channels.

23. A method of detecting an x-ray photon in computed tomography, comprising:
generating a plurality of light photons from interaction of the x-ray photon with a scintillation material;
generating an electrical signal corresponding to a number of the plurality of light photons by utilizing a silicon photomultiplier pixel; and
determining an energy of the x-ray photon based on a magnitude of the electrical signal.

24. The method of claim 23, wherein the silicon photomultiplier pixel is electrically coupled to a dedicated electronic readout channel.
